(19) United States
(12) Patent Application Publication
(10) Pub. No.: US 2026/0215871 A1
(43) Pub. Date: Jul. 30, 2026

(54) MEDICAL INSTRUMENT

(71) Applicant: Intuitive Surgical Operations, Inc., Sunnyvale, CA (US)

(72) Inventors: David I. MOREIRA RIDSDALE, Saratoga, CA (US); Wesley Chung JOE, Fremont, CA (US); Jason MIAO, Sunnyvale, CA (US); Harsukhdeep Singh RATIA, Los Altos Hills, CA (US); Ashwinram SURESH, San Jose, CA (US); Craig Keith TSUJI, San Jose, CA (US); Zhou YE, Santa Clara, CA (US); Kristopher YEE, San Jose, CA (US)

(73) Assignee: Intuitive Surgical Operations, Inc., Sunnyvale, CA (US)

(21) Appl. No.: 19/158,253

(22) PCT Filed: Feb. 21, 2024
(86) PCT No.: PCT/US2024/016719

§ 371 Date: Aug. 20, 2025

Related U.S. Application Data
(60) Provisional application No. 63/447,379, filed on Feb. 22, 2023.

(51) Int. Cl. A61B 34/00; A61B 90/00; A61B 90/70

(52) U.S. Cl. CPC A61B 34/70; A61B 90/70; A61B 2090/064

(57) ABSTRACT

A medical device includes a shaft, a beam comprising a proximal end portion coupled to a distal end portion of the shaft. A body is coupled to a distal end portion of the beam and comprises a fluid port. The medical device further includes a shroud comprising a proximal end portion, a distal end portion, and an inner wall between the proximal end portion and the distal end portion of the shroud. The distal end portion of the shroud is coupled to the body and the proximal end portion of the shroud is located between the distal end portion of the shaft and the body. The inner wall of the shroud defines an interior volume in fluid communication with the fluid port such that fluid introduced into the fluid port flows through the interior volume of the shroud and is directed proximally toward the distal end portion of the shaft.

### CROSS-REFERENCE TO RELATED APPLICATIONS

This patent application claims priority to and the filing date benefit of U.S. Provisional Ser. No. 63/447,379, filed Feb. 22, 2023, entitled “MEDICAL INSTRUMENT,” the disclosure of which is incorporated by reference herein in its entirety.

### BACKGROUND

The embodiments described herein relate to medical instruments, and more specifically to medical instruments adapted for use with teleoperated surgical systems. More particularly, the embodiments described herein relate to force sensing medical instruments that include structures to limit the range of motion of a force sensor beam to reduce force sensing artifacts that affect force feedback accuracy, and that include cleaning fluid ports and fluid routing structures adapted to support cleaning of such instruments.

Minimally Invasive Surgery (MIS) employs medical instruments that can be manually controlled or controlled via hand-held or mechanically grounded teleoperated medical systems that operate with at least partial computer assistance (“telesurgical systems”). Many known MIS instruments include a therapeutic or diagnostic end effector (e.g., forceps, a cutting tool, or a cauterizing tool) mounted on an optional wrist mechanism at the distal end of a long shaft. During an MIS procedure, the end effector, optional wrist mechanism, and the distal end of the shaft are typically inserted through a small incision or a natural orifice to position the end effector at a surgical work site within a patient. The optional wrist mechanism can be used to change the end effector's position and orientation with reference to the shaft to perform a desired procedure at the work site. Medical instruments used together with telesurgical systems typically include a proximal end mechanical structure that couples to the telesurgical system and that receives mechanical force or torque inputs used to drive the instrument's wrist and end effector components.

Force sensing medical instruments are known and, together with associated telesurgical systems, they provide force feedback sensations during a MIS procedure to a surgeon performing a procedure with such instruments. The force feedback increases the surgeon's sense of immersion, realism, and intuitiveness while performing the procedure. Various force sensing instrument architectures are known. In one example architecture, a resiliently flexible beam is coupled between the distal end of the instrument's shaft and the instrument's operative distal end components. Sensor elements mounted on the beam (e.g., strain sensors, such as Wheatstone bridge circuits and the like, optical fiber Bragg gratings, etc.) sense indications of strain in the beam as it laterally deflects due to instrument-tissue interaction, and outputs from the sensor elements are used as input for rendering force feedback sensations to the surgeon.

A mechanical hard stop structure may be used to limit the beam's lateral deflection and so protect the beam and the sensor elements, as well as to limit the sensed strain used to generate the force feedback to the surgeon. But contact between the beam and the hard stop may cause undesirable strains within the beam. As a result of these undesirable strains, the strain sensors on the beam indicate strain on the beam that does not match the actual strain on the instrument's distal end, and the force feedback to the surgeon is incorrect. This situation is further described in U.S. Patent Publication No. 2021/0353373, entitled “Hard Stop that Produces a Reactive Upon Engagement for Cantilevered-Based Force Sensing,” filed May 17, 2021, the disclosure of which is incorporated herein by reference. Therefore, improved structures for limiting lateral force sensing beam deflection are desirable. Further, the stiffness of each of the instrument's various distal end structures as they interact is important for effective force feedback rendering to the surgeon.

In addition, reusable surgical instrument exterior and interior regions must be thoroughly cleaned and sterilized. Force sensing instruments present challenges for cleaning because of the additional distal force sensing structures. Similarly, proximal end structures present challenges because of a need to restrict excess cleaning fluid from entering while interior regions of the shaft are being flushed with cleaning fluid. Therefore, improved structures for cleaning teleoperated medical instrument distal, proximal, and intermediate structures are desirable.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device includes an inner shaft, an outer shaft, and a beam. A proximal end portion of the beam is coupled to a distal end portion of the inner shaft. A body is coupled to a distal end portion of the beam and a strain sensor is coupled to the beam. A shroud optionally includes multiple slits and has a distal end portion coupled to the body. A distal end portion of the outer shaft surrounds at least a portion of the distal end portion of the inner shaft, at least a portion of the beam, and at least a portion of the shroud. The set of slits is positioned at a contact region between the shroud and the distal end portion of the outer shaft. Contact between the shroud and the distal end portion of the outer shaft limits lateral deflection of the distal end portion of the beam.

In some embodiments, each of the slits is curved. In some embodiments, each of the slits has a width less than about 0.10 mm. In some embodiments, a longitudinal axis of the shroud is defined between the proximal end portion and the distal end portion of the shroud and at the contact region, the shroud is resiliently deformable radially inward and is resiliently bendable along the longitudinal axis of the shroud.

In some embodiments, the medical device further includes a bushing, and the bushing comprises a proximal end portion and a distal end portion. The proximal end portion of the bushing is coupled to the distal end portion of the inner shaft, and the distal end portion of the bushing extends distally beyond the distal end portion of the inner shaft and over at least a portion of the beam. The outer shaft extends over and is in sliding contact with the bushing.

In some embodiments, the shroud comprises a tab and the shroud is coupled to the body by the tab captured between the body and the distal end portion of the beam. In some embodiments, the inner shaft translates within the outer shaft. In some embodiments, the inner shaft translates within the outer shaft within a range of motion defined between a proximal range of motion limit and a distal range of motion limit. The proximal end portion of the shroud remains within the outer shaft within the range of motion of the inner shaft.

In some embodiments, a medical device includes a shaft comprising a distal end portion, a beam comprising a proximal end portion and a distal end portion, and the proximal end portion of the beam is coupled to the distal end portion of the shaft. A body is coupled to the distal end portion of the beam and comprises a fluid port. The medical device further includes a shroud comprising a proximal end portion, a distal end portion, and an inner wall between the proximal end portion and the distal end portion of the shroud. The distal end portion of the shroud is coupled to the body and the proximal end portion of the shroud is located between the distal end portion of the shaft and the body. The inner wall of the shroud defines an interior volume in fluid communication with the fluid port such that fluid introduced into the fluid port flows through the interior volume of the shroud and is directed proximally toward the distal end portion of the shaft.

In some embodiments, the medical device includes an end effector actuator element. that extends through the distal end portion of the shaft and exits the distal end portion of the shaft at an exit location. Fluid introduced into the fluid port flows through the interior volume of the shroud, is directed proximally along the actuator component, and is directed against the exit location.

In some embodiments, the medical device further comprises a bushing having a proximal end portion and a distal end portion. The proximal end portion of the bushing is coupled to the distal end portion of the shaft, and the distal end portion of the bushing is located between the distal end portion of the shaft and the proximal end portion of the shroud.

In some embodiments, the bushing defines an interior volume between the distal end portion of the shaft and the distal end portion of the bushing, and fluid introduced into the fluid port flows through the interior volume of the shroud and proximally into the interior volume of the bushing.

In some embodiments, the medical device includes an end effector actuator element, and the bushing defines an interior volume between the distal end portion of the shaft and the distal end portion of the bushing. The end effector actuator element extends through the distal end portion of the shaft, exits the distal end portion of the shaft at an exit location, and extends through the interior volume of the bushing. Fluid introduced into the fluid port flows through the interior volume of the shroud, is directed proximally along the end effector actuator element, is directed into the interior volume of the bushing, and is directed against the exit location.

In some embodiments, the shaft is an inner shaft, and the medical device further comprises an outer shaft surrounding at least a portion of the inner shaft. The outer shaft comprises a distal end, and the shroud optionally comprises a set of slits positioned at a contact region between the proximal end portion of the shroud and the distal end of the outer shaft. Contact between the proximal end portion of the shroud and the distal end of the outer shaft limits lateral deflection of the distal end portion of the beam.

In some embodiments, a longitudinal axis of the shroud is defined between the proximal end portion and the distal end portion of the shroud, and at the contact region, the shroud is resiliently deformable radially inward and is resiliently bendable along the longitudinal axis of the shroud.

In some embodiments, each slit of the set of slits is shaped, sized, or shaped and sized to restrict capture of a surgical suture. In some embodiments, the shaft is an inner shaft, and the medical device further comprises an outer shaft surrounding at least a portion of the inner shaft and surrounding the proximal end portion of the shroud. The inner shaft translates within the outer shaft within a range of motion defined between a proximal range of motion limit and a distal range of motion limit. The proximal end portion of the shroud remains within the outer shaft within the range of motion of the inner shaft.

In some embodiments, a medical device includes a fluid routing structure, a shaft extending within at least a portion of the fluid routing structure and a flow restriction tube surrounding the shaft within the fluid routing structure. A flow restriction tube stop is positioned proximally of the flow restriction tube. Pressure from a fluid introduced against the flow restriction tube causes the flow restriction tube to translate proximally with reference to the shaft until the flow restriction tube contacts the flow restriction tube stop. Contact between the flow restriction tube and the flow restriction tube stop restricts the fluid from traveling proximally past the flow restriction tube stop.

In some embodiments, the fluid routing structure comprises a flush port structure that defines a flush port. The fluid introduced against the flow restriction tube is introduced through the flush port and thereafter routed proximally along an exterior surface of the shaft. In some embodiments, the flow restriction tube is positioned to translate along a length of the shaft within the fluid routing structure.

In some embodiments, the shaft is an inner shaft, and the medical device further comprises an outer shaft and a coupler. The outer shaft comprises a proximal end portion coupled to the coupler. The coupler is coupled to the fluid routing structure and comprises a port in fluid communication with the flush port of the fluid port structure. In some embodiments, the fluid introduced against the flow restriction tube is introduced through the flush port and routed through the port of the coupler and distally between the exterior surface of the shaft and an interior surface of the outer shaft.

In some embodiments, the flush port is a first flush port, the medical device further comprises a proximal mechanical structure coupled to the fluid routing structure, and the flush port structure comprises a second flush port. A fluid introduced into the second flush port is directed proximally to a location within the proximal mechanical structure. In some embodiments, the coupler comprises a proximal end, a longitudinal axis of the flow restriction tube is defined between a proximal end of the flow restriction tube and a distal end of the flow restriction tube, and the flow restriction tube is movable along the longitudinal axis of the flow restriction tube between the proximal end of the coupler and the flow restriction tube stop.

### BRIEF DESCRIPTION OF THE DRAWINGS

FIG. 1 is a plan view of a minimally invasive teleoperated surgery system according to an embodiment being used to perform a medical procedure such as surgery.

FIG. 2 is a perspective view of a user control console of the minimally invasive teleoperated surgery system shown in FIG. 1.

FIG. 3 is a perspective view of an optional auxiliary unit of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5 is a diagrammatic illustration of a medical device, according to an embodiment.

FIG. 6 is a diagrammatic illustration of a medical device, according to an embodiment.

FIG. 7A is a diagrammatic illustration of a medical device, according to an embodiment, shown with a fluid restriction tube in a first position.

FIG. 7B is a diagrammatic illustration of the medical device of FIG. 7A, shown with the fluid restriction tube in a second position.

FIG. 8 is a perspective view of a medical device, according to another embodiment.

FIG. 9 is an enlarged perspective view of a distal end portion of the medical device of FIG. 8.

FIG. 10 is a side view of a distal end portion of the medical device of FIG. 8 with the outer shaft shown in transparency for illustration purposes.

FIG. 11A is a perspective view of a portion of the medical device of FIG. 8 with the outer shaft removed and the distal bushing shown in transparency for illustration purposes.

FIG. 11B is a side view of a portion of the medical device of FIG. 8 with the outer shaft removed and the distal bushing shown in transparency for illustration purposes.

FIG. 11C is an end perspective view of the distal bushing of the medical device of FIG.

FIG. 12A is a perspective view of a distal end portion of the medical device of FIG. 8 with select components removed for illustration purposes.

FIG. 12B is a perspective view of a distal end portion of the medical device of FIG. 8 with select components removed for illustration purposes.

FIG. 13 is an exploded perspective view of the portion of the medical device of FIG. 12.

FIG. 14 is a perspective view of the beam with outer mold of the medical device of FIG. 8.

FIG. 15A is a perspective view of the shroud of the medical device of FIG. 8.

FIG. 15B is a perspective view of a link of the medical device of FIG. 8.

FIG. 16 is a perspective view of a proximal end portion of medical device of FIG. 8, with select components of the proximal mechanical structure removed for illustration purposes.

FIG. 17 is an exploded view of a proximal end portion of the medical device of FIG. 8 with select components removed for illustration purposes.

FIG. 18 is a cross-sectional view of the proximal end portion of the medical device of FIG. 16 taken along line 18-18.

FIG. 19 is a cross-sectional view of the proximal end portion of the medical device of FIG. 16 taken along line 19-19.

FIG. 20 is a cross-sectional view of the proximal end portion of the medical device of FIG. 16 taken along the line 20-20.

### DETAILED DESCRIPTION

The embodiments described herein can advantageously be used in a wide variety of force sensing instrument applications, such as for grasping, cutting, and manipulating operations associated with minimally invasive surgery. The embodiments described herein can also be used in a variety of non-medical applications such as, for example, teleoperated systems for search and rescue, remotely controlled submersible devices, aerial devices, automobiles, etc. The medical instruments or devices of the present application enable motion in three or more degrees of freedom (DOFs). For example, in some embodiments, an end effector of the medical instrument can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF). Thus, in some embodiments, the medical instruments or devices of the present application enable motion in six DOFs. The embodiments described herein can further be used to determine the forces exerted on (or by) a distal end portion of the instrument during use.

Embodiments described herein relate to force sensing medical instruments for determining forces applied to the medical instrument to control a surgical system, such as a minimally invasive teleoperated surgery system. In some embodiments described herein, the medical instruments include one or more flush ports at a distal end of the instrument, one or more flush ports at a proximal end of the instrument or both. In some embodiments described herein, structures are provided at a distal end portion of the instrument to limit the range of motion of a force sensor beam and reduce force artifacts that affect the accuracy of force feedback.

In some embodiments, a force sensing medical instrument includes a force sensor system that includes a distal force sensor unit that can provide an indication of forces affecting the instrument. This indication of the force(s) can be used by the system to deliver force feedback to a user control unit of the system. The distal force sensor unit can include a strain sensor coupled to a resiliently deformable beam. The beam is configured to deform in response to a load affecting at a distal end portion of the instrument. The strain sensor includes one or more strain gauges that measure the resultant strain in the beam due to the deflection. In some embodiments, a sensor signal cable can be coupled to the distal force sensor unit, extend proximally, and be coupled to an electronic circuit board of the medical device. Such an electronic circuit board is described in detail in co-pending U.S. Provisional Patent Application No. 63/425,524, filed on Nov. 15, 2022, the disclosure of which is incorporated herein by reference. The sensor signal cable carries the strain signal to the electronic circuit board. Further details regarding the sensor signal cable are provided in co-pending U.S. Provisional Patent Application No. 63/425,520, filed on Nov. 15, 2022, the disclosure of which is incorporated herein by reference.

In some embodiments, medical devices described herein include a force sensor unit having a beam and one or more strain sensors on the beam. The medical devices include a shroud that surrounds at least a portion of the beam and is coupled to the beam. The shroud is formed with a super elastic shape-memory material and optionally includes multiple slits along a wall of the shroud. The material and/or slits (when formed or otherwise included on the shroud) allow the shroud to be resiliently bendable along a longitudinal axis of the shroud and resiliently deformable radially inward. An outer shaft surrounds at least a portion of the shroud and has a distal end portion that is positioned such that the slits of the shroud are positioned at a contact region between the shroud and the distal end portion of the outer shaft. During use of the medical device, contact between the shroud and the distal end portion of the outer shaft can limit lateral deflection of the distal end portion of the beam while also limiting distortion of the sensed forces. For example, because the shroud is coupled to the beam, as the beam bends due to forces exerted on the distal end portion of the medical device, the shroud will move with the beam, until it contacts the outer shaft. The resiliency of the shroud allows the shroud to deform or bend as it contacts the outer shaft, and then revert to its original linear shape. In other words, the shroud has a biased linear shape and can bend or deform through contact with the outer shaft and revert to its biased linear shape when there is no longer contact with the outer shaft. Deformation of the shroud allows for limitation of the deflection of the beam while also limiting the distortion of the sensed forces. Similarly stated, the slits produce a deformation region that has a stiffness that is much smaller than the stiffness of the beam, thereby limiting the distortion of the sensed forces.

In some embodiments, medical devices are described herein that include a fluid flush port at a distal end portion of the medical device. The distal flush port provides for cleaning fluid to be introduced into the interior of the medical device to provide effective cleaning of the interior components that may otherwise be blocked from access. The shroud can function to deflect or direct the fluid proximally from the distal fluid port. In some embodiments, distal flush port is located on a body component coupled to a distal end portion of the shroud. The body can be, for example, a link of a wrist assembly or component of an end effector. The size and the location of the distal flush port are selected to enable easy access and connection to a luer fitting to connect a fluid source to the medical device.

In some embodiments, medical devices are described herein that provide one or more flush ports at a proximal end portion of the medical device. For example, a first flush port can allow for introduction of cleaning fluid into an interior of the medical device at a proximal end portion of the inner shaft and fluid can be directed distally along an exterior surface of the shaft between the exterior surface of the shaft and an interior surface of an outer shaft surrounding the inner shaft. When fluid is introduced into the medical device through the flush port, some fluid may be directed proximally. A fluid restriction tube and fluid restriction tube stop function to restrict the fluid from flowing proximally past the fluid restriction tube stop. The medical device can include a second flush port at the proximal end portion that can be used to introduce cleaning fluid into the medical device that is then directed proximally into an interior of a proximal mechanical structure coupled to proximal end portion of the inner shaft.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

The term “flexible” in association with a part, such as a mechanical structure, component, or component assembly, should be broadly construed. In essence, the term means the part can be repeatedly bent and restored to an original shape without harm to the part. Certain flexible components can also be resilient. For example, a component (e.g., a flexure) is said to be resilient if possesses the ability to absorb energy when it is deformed elastically, and then release the stored energy upon unloading (i.e., returning to its original state). Many “rigid” objects have a slight inherent resilient “bendiness” due to material properties, although such objects are not considered “flexible” as the term is used herein.

As used in this specification and the appended claims, the word “distal” refers to direction towards a work site, and the word “proximal” refers to a direction away from the work site. Thus, for example, the end of a tool that is closest to the target tissue would be the distal end of the tool, and the end opposite the distal end (i.e., the end manipulated by the user or coupled to the actuation shaft) would be the proximal end of the tool.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like-may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes include various spatial device positions and orientations. The combination of a body's position and orientation defines the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, instrument, and variants thereof, can be interchangeably used.

Inventive aspects are described with reference to a teleoperated surgical system. An example architecture of such a teleoperated surgical system is the da Vinci® surgical system commercialized by Intuitive Surgical, Inc., Sunnyvale, California. Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations are merely presented as examples, and they are not to be considered as limiting the scope of the inventive aspects disclosed herein. As applicable, inventive aspects may be embodied and implemented in both relatively smaller, hand-held, hand-operated devices and relatively larger systems that have additional mechanical support.

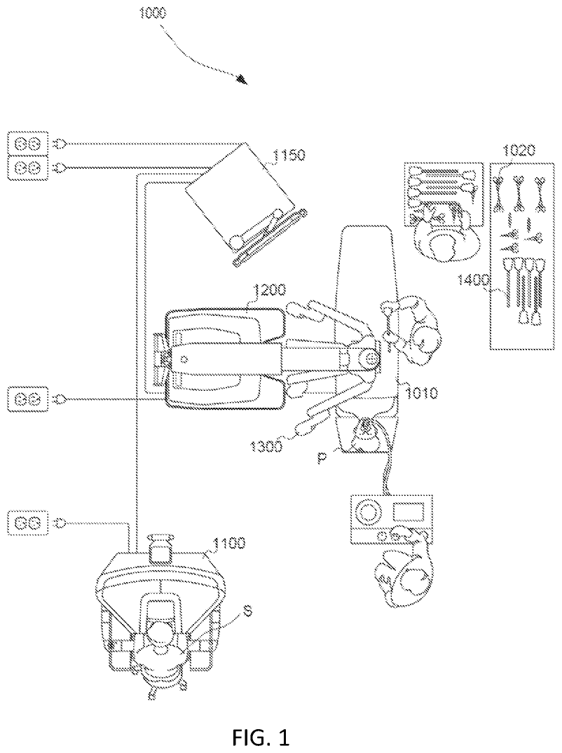

FIG. 1 is a plan view illustration of a teleoperated surgical system (“system”) 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by an operator of the system, such as a surgeon or other skilled clinician S, during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled medical instrument (instrument) 1400 (e.g., a force sensing medical instrument) through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the system 1000.

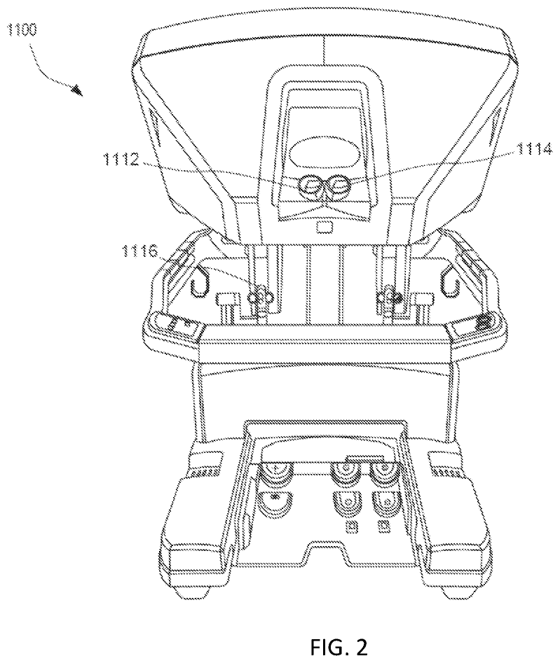

FIG. 2 is a perspective view of the user control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116 (input device), which in turn causes the manipulator unit 1200 (shown in FIG. 1) to manipulate one or more tools. The input devices 1116 provide at least the same degrees of freedom as instruments 1400 with which they are associated to provide the surgeon S with telepresence, or the perception that the input devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

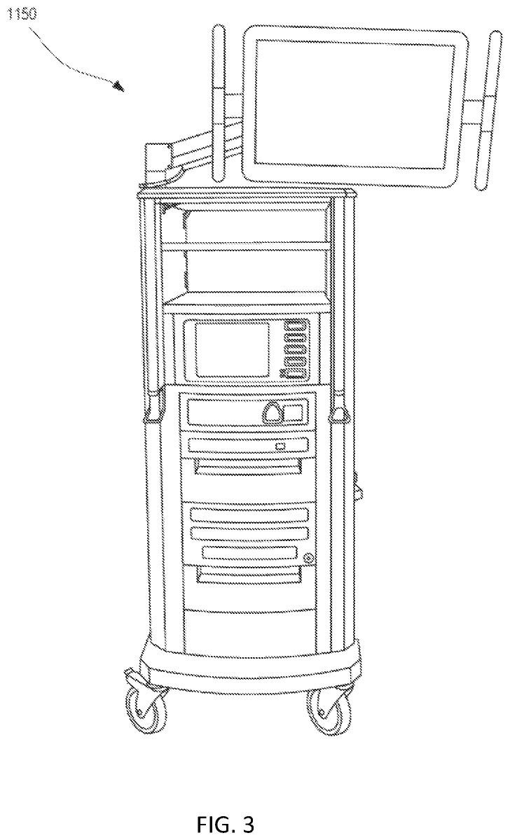

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

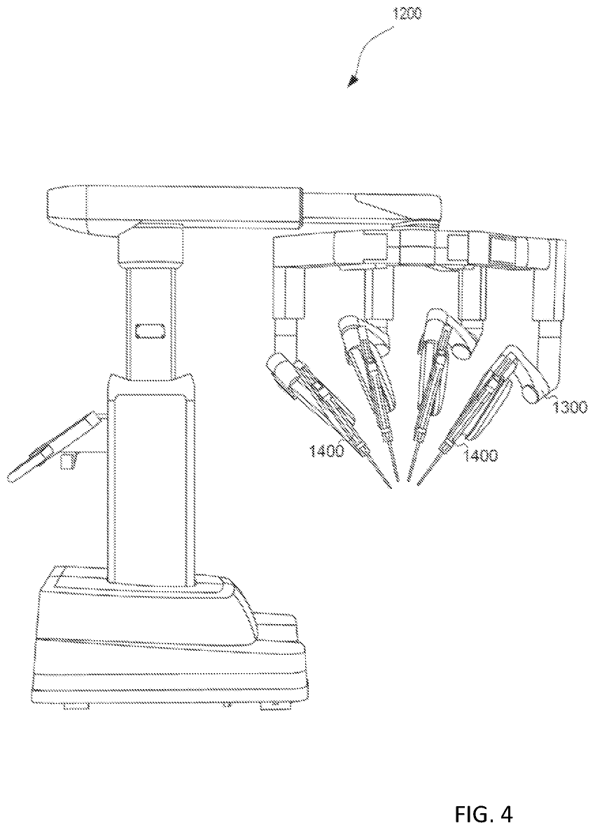

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

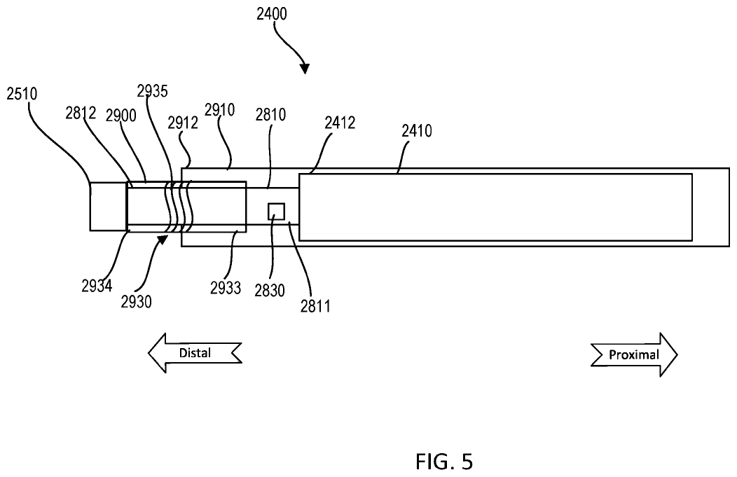

FIG. 5 is a schematic illustration of a medical device 2400, according to an embodiment. In some embodiments, the medical device 2400 or any of the components therein are optionally parts of a surgical system that performs surgical procedures, and which can include a manipulator unit, a series of kinematic linkages, a series of cannulas, or the like. The medical device 2400 (and any of the instruments described herein) can be used in any suitable surgical system, such as the MIRS system 1000 shown and described above. The medical device 2400 includes an inner shaft 2410, an outer shaft 2910, a beam 2810, a shroud 2900 and a body 2510. The inner shaft 2410 includes a distal end portion 2412 coupled to a proximal end portion 2811 of the beam 2810. The body 2510 is coupled to a distal end portion 2812 of the beam 2810. The shroud 2900 includes a distal end portion 2934 coupled to the body 2510 and to the beam 2810 and a proximal end portion 2933 that extends proximally over the beam 2810. In some embodiments, the shroud 2900 includes a tab (not shown in FIG. 5) that is captured between the body 2510 and the distal end portion 2812 of the beam 2810 to couple the shroud 2900 to the body 2510 and the beam 2810.

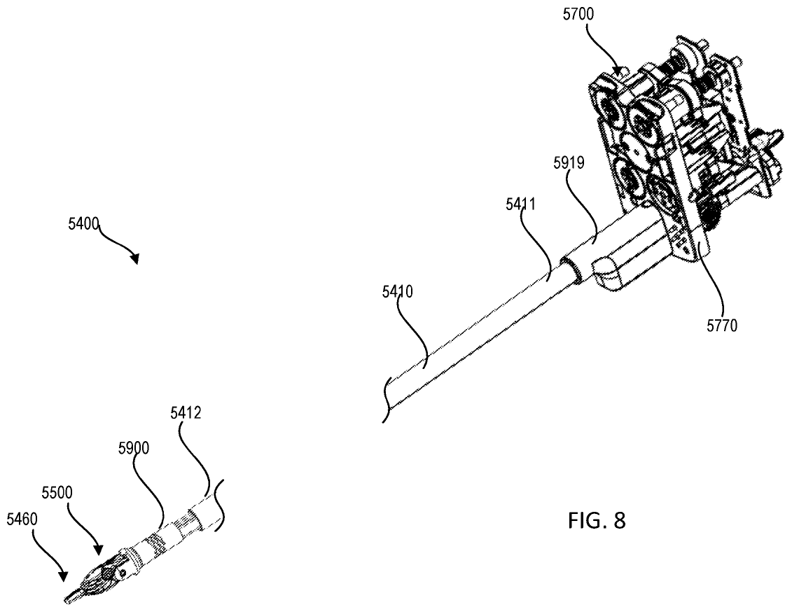

The beam 2810 is part of a force sensor system of the medical device 2400 that includes at least one strain sensor 2830 positioned on the beam 2810. Generally, during a medical procedure, the tool of the medical device 2400 contacts anatomical tissue, which may result in x and y direction forces, which can be radial, transverse, or perpendicular to the shaft's long axis or z direction forces, which are axial or parallel to the shaft's long axis (see, e.g., x, y, and z axes directions shown in FIG. 8) being imparted on the tool. The strain sensor 2830 can measure strain in the beam 2810 during operation of the medical device 2400. The measured beam strain can be used to determine forces imparted on the tool in the x- and y-axis directions. These x- and y-axis forces are transverse (e.g., perpendicular) to the z-axis (which is parallel or collinear with a center axis of the beam).

In some embodiments, the body 2510 can be a link included within a wrist assembly, which has multiple articulating links. In some embodiments, an end effector including a tool (not shown) is coupled to the body 2510 (or to a wrist assembly) at a distal end portion of the medical device. The tool can include, for example, articulatable jaws or another suitable surgical tool that is coupled to the body 2510. An end effector actuator element (not shown) can be coupled to the body 2510 and to the tool and can be, for example, a cable, band, rod or the like. The end effector actuator element can extend through the inner shaft 2410 and be coupled to a mechanical structure (not shown in FIG. 5). The mechanical structure can include components configured to actuate the end effector actuator element, which causes one or more components of the surgical instrument to move, such as, for example, the tool. In some embodiments, a mechanical structure can be configured similar to or the same as the proximal mechanical structure 5700 described below.

In some embodiments, the shroud 2900 optionally includes multiple slits 2935. The slits 2935 are merely optional design features that can provide certain improvements, but are not required to be included in any of the embodiments as described herein. The multiple slits 2935 are defined through a wall of the shroud 2900. In the depicted example, the slits 2935 have a wavy or curved shape, and have a width sized to prevent sutures catching in the slits 2935. This can be particularly advantageous in applications where the end effector is a needle driver for use in suturing during various procedures. With such a configuration of the slits 2935, even during deformation of the shroud, the shroud 2900 can avoid any undesirable pinching or catching of the suture. In some embodiments, the slits 2935 have a width of less than about 0.10 mm. The shroud is positioned to cover and protect the strain sensor 2830 on the beam 2810, along with actuation elements, wires, etc. that may be located at the distal end portion of the medical device 2400. The shroud can also cover and protect actuator elements (e.g., drive cables) or cautery wires, etc. The shroud 2900 can be formed with a super elastic, shape-memory material, such as, for example, a nickel titanium alloy (e.g., Nitinol alloy), such that deformation or bending of the shroud 2900 is not permanent. In other words, the shroud 2900 is resiliently deformable radially inward and resiliently bendable radially inward during use of the medical device 2400, as described in more detail below. The super elastic material of the shroud 2900 provides more tolerance for misalignment between the shroud 2900 and the inner shaft 2410 due to its flexibility, which also provides more sensing range for the force sensor unit. The shroud 2900 can also be formed with a thinner wall thickness to enhance the sensing range. For example, in some embodiments, the wall thickness of the shroud 2900 can be 0.076 mm (0.003 inches), providing more clearance between the inner shaft 2410 and the shroud 2900, enhanced sensing range, and more space for cleaning the medical device (described in more detail below). The shroud 2900 can also deflect during cleaning for better flow of fluid within the medical device 2400.

The outer shaft 2910 extends distally over and surrounds the distal end portion 2412 of the inner shaft 2410, a portion of the beam 2810, and a portion of the shroud 2900, such that a distal end portion 2912 of the outer shaft 2910 is positioned at a contact region 2930 between the distal end portion 2912 of the outer shaft 2910 and the shroud 2900. As shown in FIG. 5, the contact region 2930 is associated with the location of the multiple slits 2935 (e.g., the contact region is at a same or generally same location as the slits 2935) when the shroud 2900 includes such slits 2935. The outer shaft 2910 extending partially over the slits 2935 also minimizes exposure of the slits 2935 to fluids and other bodily materials during use. In some embodiments, the inner shaft 2410 translates within the outer shaft 2910 within a range of motion defined between a proximal range of motion limit and a distal range of motion limit. In such an embodiment, the proximal end portion 2933 of the shroud 2900 remains within the outer shaft 2910 within the range of motion of the inner shaft 2410.

During operation of the medical device, contact between the shroud 2900 and the distal end portion 2912 of the outer shaft 2910 can limit lateral deflection of the distal end portion of the beam. For example, during operation of the medical device 2400, if the beam 2810 is caused to bend due to outside forces imparted on the distal end portion of the medical device 2400 (e.g., on the body 2510), the beam 2810 can bend radially outward. Because the shroud 2900 is coupled to the beam 2800, as the beam 2800 bends due to these outside forces, the shroud 2900 will move with the beam 2810 until it contacts the outer shaft 2910. The material, thin wall thickness and slits of the shroud 2900 allow the shroud 2900 to resiliently deform radially inward and/or resiliently bend along the longitudinal axis of the shroud 2900 as it contacts the outer shaft 2910. In doing so, the lateral deflection of the beam 2810 is limited by the contact between the shroud 2900 and the outer shaft 2910. As stated above, the shroud 2900 can then revert to its biased linear shape when no longer in contact with the outer shaft 2910.

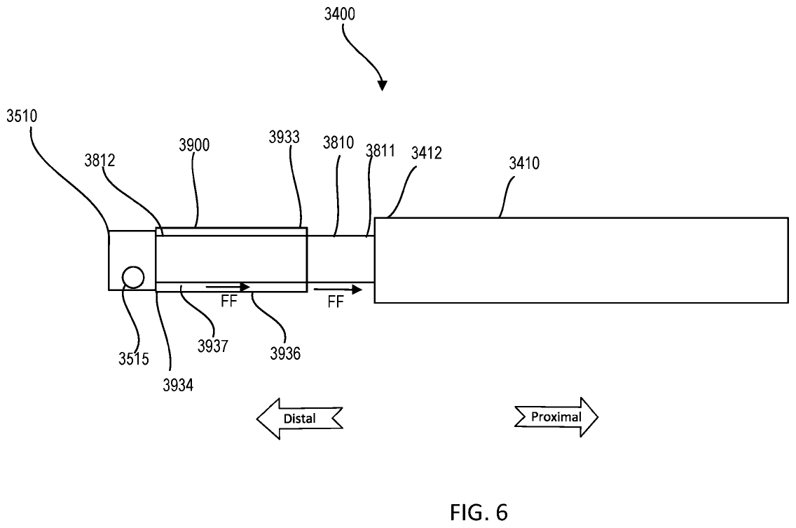

FIG. 6 is a schematic illustration of portion of a medical device 3400, according to another embodiment. In some embodiments, the medical device 3400 or any of the components therein are optionally parts of a surgical system that performs surgical procedures, and which can include a manipulator unit, a series of kinematic linkages, a series of cannulas, or the like. The medical device 3400 (and any of the instruments described herein) can be used in any suitable surgical system, such as the MIRS system 1000 shown and described above. The medical device 3400 includes a shaft 3410, a beam 3810, a shroud 3900 and a body 3510. The shaft 3410 includes a distal end portion 3412 coupled to a proximal end portion 3811 of the beam 3810. The body 3510 is coupled to a distal end portion 3812 of the beam 3810. The shroud 3900 includes a distal end portion 3934 coupled to the body 3510 and to the beam 3810 and a proximal end portion 3933 that extends proximally over the beam 3810. In some embodiments, the shroud 3900 includes a tab (not shown in FIG. 6) that is captured between the body 3510 and the distal end portion 3812 of the beam 3810 to couple the shroud 3900 to the body 3510 and the beam 3810.

The beam 3810 is part of a force sensor system of the medical device 3400 that includes at least one strain sensor (not shown in FIG. 6) positioned on the beam 3810. As described above, during a medical procedure, the tool of the medical device 3400 contacts anatomical tissue, which may result in x and y direction forces, which can be radial, transverse, or perpendicular to the shaft's long axis or z direction forces, which are axial or parallel to the shaft's long axis (see, e.g., x, y, and z axes directions shown in FIG. 8) being imparted on the tool. The strain sensor can measure strain in the beam 3810 during operation of the medical device 3400. The measured beam strain can be used to determine forces imparted on the tool in the x- and y-axis directions. These x- and y-axis forces are transverse (e.g., perpendicular) to the z-axis (which is parallel or collinear with a center axis of the beam).

As shown in FIG. 6, the body 3510 includes a fluid port 3515 through which fluid can be introduced into the medical device 3400 to clean interior components of the medical device 3400, as described in more detail herein. In some embodiments, the body 3510 is a link included within a wrist assembly, which has multiple articulating links. In some embodiments, an end effector including a tool (not shown) is coupled to the body 3510 at a distal end portion of the medical device. The tool can include, for example, articulatable jaws or another suitable surgical tool that is coupled to the body 3510. An end effector actuator element (not shown) can be coupled to the body 3510 and to the tool and can be, for example, a cable, band, rod or the like. The end effector actuator element can extend through the shaft 3410 and exit the shaft 3410 at an exit location and be coupled to a mechanical structure (not shown in FIG. 6). The mechanical structure can include components configured to actuate the end effector actuator element, which causes one or more components of the surgical instrument to move, such as, for example, the tool. In some embodiments, a mechanical structure can be configured similar to or the same as, the proximal mechanical structure 5700 described below.

The shroud 3900 has an inner wall 3936 between the proximal end portion 3933 and the distal end portion 3934 of the shroud 3900. The inner wall 3936 of the shroud 3900 defines an interior volume 3937 in fluid communication with the fluid port 3515 of the body 3510 such that fluid introduced into the fluid port 3515 flows through the interior volume 3937 of the shroud 3900 and is directed proximally toward the distal end portion 3412 of the shaft 3410 as shown by arrows FF in FIG. 6. In some embodiments, fluid introduced into the fluid port 3515 flows through the interior volume 3937 of the shroud 3900, is directed proximally along the end effector actuator element, and is directed against the exit location of the end effector actuator element. The ability to introduce fluid into the distal end of the medical device 2400 is important as fluid introduced from a proximal end of the medical device 2400 may be prevented form flowing distally to the distal end of the medical device due to interior components obstructing the flow.

In some embodiments, as described above for shroud 2900, the shroud 3900 can be formed with a super elastic, shape-memory material, such as, for example, Nitinol alloy. The super elastic material of the shroud 3900 provides more tolerance for misalignment between the shroud 3900 and the shaft 3410 due to the flexibility of the shroud 3900 providing more sensing range. The shroud 3900 can also be formed with a thinner wall thickness to enhance the sensing range. For example, in some embodiments, the wall thickness of the shroud 3900 can be 0.076 mm (0.003 inches), providing more clearance between the shaft 3410 and the and the shroud 3900, enhanced sensing range, and more space for cleaning the medical device (described in more detail below) and can deflect during cleaning for better flow of fluid within the medical device 3400. The shape-memory aspects of the Nitinol alloy material of the shroud 3900 provides the shroud 3900 with a lower elastic modulus than, for example, a stainless steel, which will allow for the shroud 3900 to avoid permanent deformation at high strains.

In some embodiments, the medical device 3400 can optionally include a bushing (not shown in FIG. 6) having a proximal end portion coupled to the distal end portion 3412 of the shaft 3410, and a distal end portion located between the distal end portion 3412 of the shaft 3410 and the proximal end portion 3933 of the shroud 3900. The bushing can define an interior volume between the distal end portion 3412 of the shaft 3410 and the distal end portion of the bushing, and fluid introduced into the fluid port 3515 of the body 3510 flows through the interior volume 3937 of the shroud 3900 and proximally into the interior volume of the bushing. In some embodiments, the medical device 3400 includes an end effector actuator element (not shown in FIG. 6) that extends through the shaft 3410, exits the distal end portion 3412 of the shaft 3410 at an exit location, and extends through the interior volume of the bushing. In some embodiments fluid introduced into the fluid port 3515 flows through the interior volume 3937 of the shroud 3900, is directed proximally along the end effector actuator element, is directed into the interior volume of the bushing, and is directed against the exit location.

In some embodiments, the medical device 3400 includes an outer shaft (not shown in FIG. 6) that surrounds the distal end portion 3412 of the shaft 3410, at least a portion of the shroud 3900 and at least a portion of the bushing. In some embodiments, the proximal end portion 3933 of the shroud 3900 is positioned proximally of a distal end portion of the outer shaft such that fluid introduced through the flush port 3515 is directed proximally through the interior volume 3937 of the shroud 3900, and the shroud 3900 helps direct the fluid proximally to within the interior volume of the bushing. Thus, the outer shaft surrounds the proximal end portion 3933 of the shroud 3900 such that the fluid is directed to the distal end portion of the distal bushing.

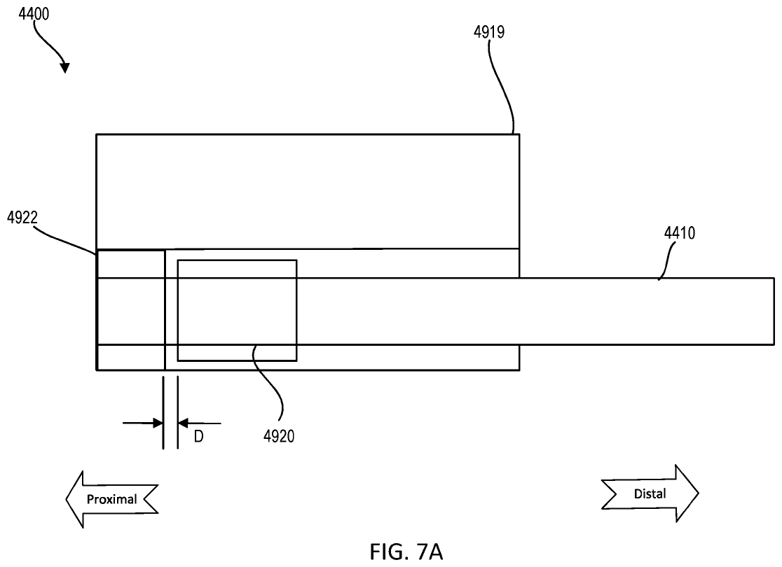

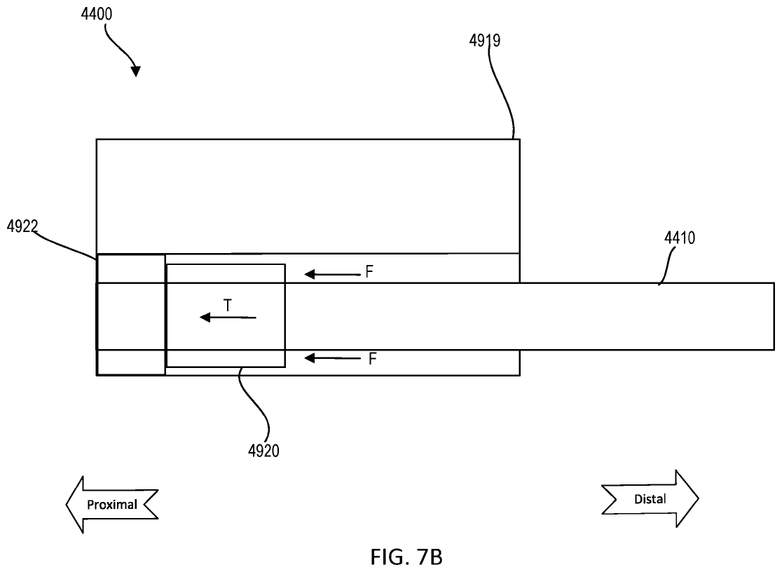

FIGS. 7A and 7B are schematic illustrations of portion of a medical device 4400, according to another embodiment. In some embodiments, the medical device 4400 or any of the components therein are optionally parts of a surgical system that performs surgical procedures, and which can include a manipulator unit, a series of kinematic linkages, a series of cannulas, or the like. The medical device 4400 (and any of the instruments described herein) can be used in any suitable surgical system, such as the MIRS system 1000 shown and described above. The medical device 4400 includes a shaft 4410, a fluid routing structure 4919, a flow restriction tube 4920 and a flow restriction tube stop 4922. The shaft 4410 extends within at least a portion of the fluid routing structure 4919 and the flow restriction tube 4920 surrounds a portion of the shaft 4410 within the flow restriction structure 4919. The flow restriction tube stop 4922 is positioned proximally of the flow restriction tube 4920. The fluid routing structure 4919 allows for the introduction of fluid into the medical device 4400 to clean interior components of the medical device 4400. When a fluid is introduced into the fluid routing structure 4919 at a location distally of the flow restriction tube 4920, a pressure force F from the fluid against the flow restriction tube 4920 causes the flow restriction tube 4920 to translate proximally with reference to the shaft 4410 until the flow restriction tube 4920 contacts the flow restriction tube stop 4922 limiting the travel of the flow restriction tube 4920. As shown in FIG. 7A, in a first position, the flow restriction tube 4920 is spaced from the flow restriction tube stop 4922 a distance D. As shown in FIG. 7B, when fluid pressure force F is introduced against a distal end of the flow restriction tube 4920, the flow restriction tube 4920 translates proximally (see arrow T) along a length of the shaft 4410 until it contacts the flow restriction tube stop 4922. The flow restriction tube stop 4922 is sized and shaped to restrict the fluid from traveling proximally past the flow restriction tube stop 4922. In this manner, the fluid is forced to travel distally, as desired to produce the desired cleaning effect.

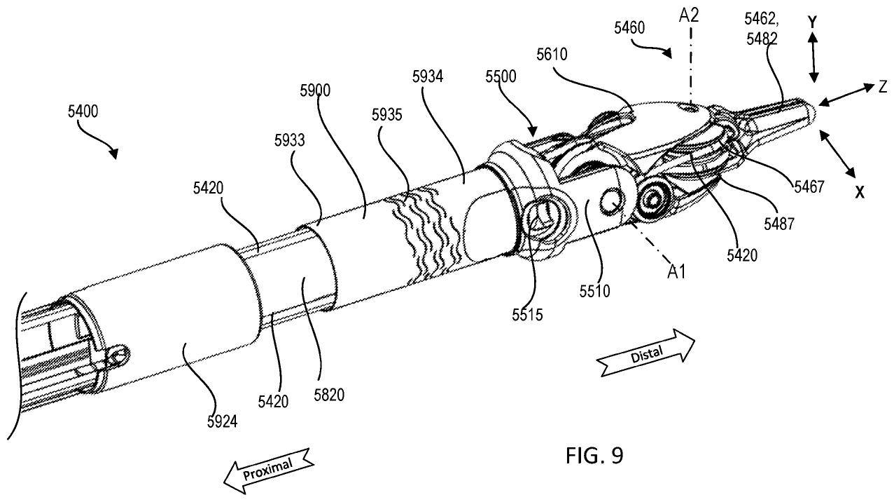

The flow restriction tube 4920 has an inner diameter that is sized to have a small gap to an outer surface of the shaft 4410, such that the shaft 4410 is free to translate longitudinally in a z-direction (see FIG. 9) (i.e., proximally and distally). When no fluid pressure is present, the flow restriction tube 4920 has a close fit to the shaft 4410, but is free floating, while the flow restriction tube stop 4922 has a large gap to the outer surface of the shaft 4410 and is in a fixed position and does not move. When the pressure of fluid against the flow restriction tube 4920 is present, the flow restriction tube 4920 closes the gap between the flow restriction tube 4920 and the flow restriction tube stop 4922. Because the gap between the flow restriction tube 4920 and the shaft 4410 is small, there is no path for the fluid to flow (e.g., the fluid is forced to travel distally as described above).

In some embodiments, the fluid routing structure 4919 includes a flush port structure (not shown in FIGS. 7A and 7B), and the fluid flush port structure can include one or more fluid ports through which a fluid can be introduced and directed proximally along an exterior of the shaft 4410, and proximally against the distal end of the flow restriction tube 4920. In some embodiments, the medical device 4400 further includes an outer shaft with a proximal end portion coupled to a coupler (each not shown in FIGS. 7A and 7B). The coupler is coupled to the fluid routing structure 4919 and includes a fluid port in fluid communication with the flush port of the fluid port structure and fluid introduced against the flow restriction tube 4920 is introduced through the flush port of the fluid port structure and routed through the port of the coupler and distally between the exterior surface of the shaft and an interior surface of the outer shaft. In some embodiments, a longitudinal axis of the flow restriction tube 4920 is defined between a proximal end of the flow restriction tube 4920 and a distal end of the flow restriction tube 4920 and the flow restriction tube 4920 is movable along the longitudinal axis of the flow restriction tube 4920 between a proximal end of the coupler and the flow restriction tube stop 4922.

In some embodiments, the flow routing structure 4919 includes a flush port structure that includes a first flush port and a second flush port. Fluid introduced into the first flush port is directed proximally toward the flow restriction tube 4920 and distally between the exterior surface of the shaft and the interior surface of an outer shaft. Fluid introduced into the second flush port is directed proximally to a location within a mechanical structure coupled to the fluid routing structure 4919. Thus, the first flush port and the second flush port are not in fluid communication with each other.

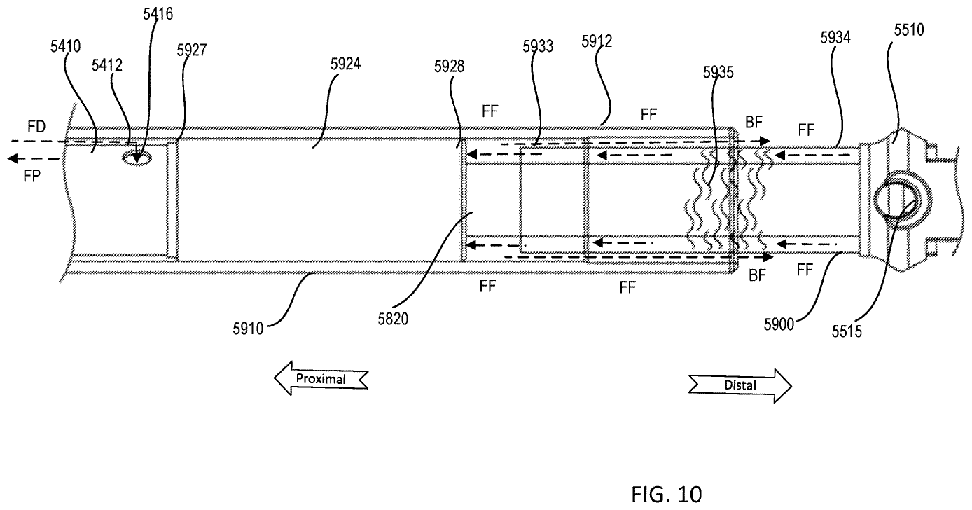

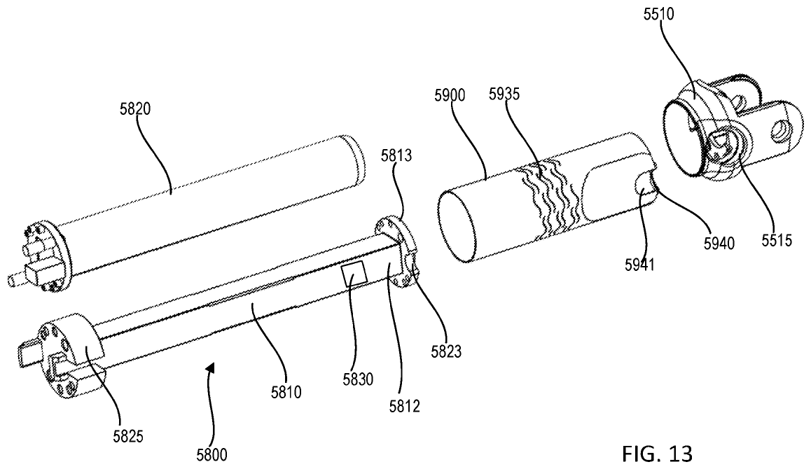

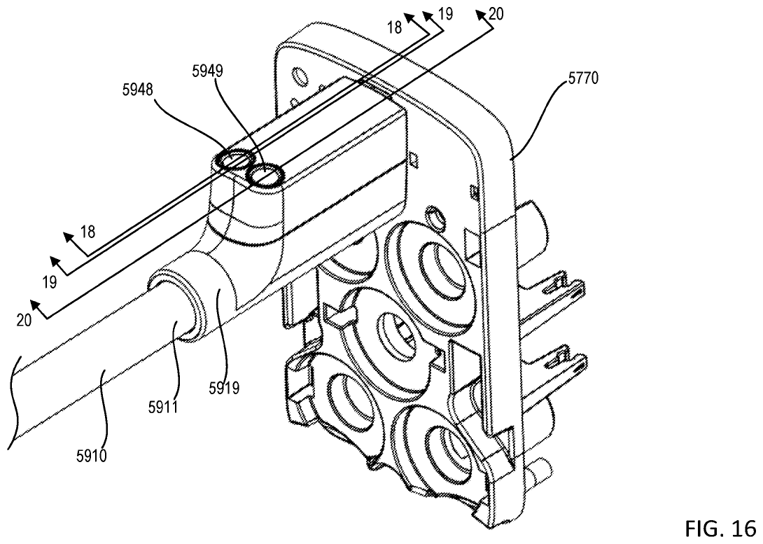

FIGS. 8-20 illustrate a medical device 5400, according to another embodiment. The medical device 5400 includes various components as described above for previous embodiments that provide for improved accuracy in force sensing and fluid flush capabilities. In some embodiments, the medical device 5400 or any of the components therein are optionally parts of a surgical system that performs surgical procedures, and which can include a manipulator unit, one or more kinematic linkages, one or more cannulas, or the like. The medical device 5400 (and any of the instruments described herein) can be used in any suitable surgical system, such as the MIRS system 1000 shown and described above. As shown in FIGS. 8-15, the medical device 5400 includes an outer shaft 5910 (see, e.g., FIG. 10), a proximal mechanical structure 5700, a fluid routing structure 5919, a distal force sensor unit 5800 including a beam 5810 (see FIG. 13), a distal bushing 5924, a shroud 5900, an inner shaft 5410, a wrist assembly 5500, and an end effector 5460 at a distal end portion of the medical device 5400. The fluid routing structure 5919 includes fluid routing components at a proximal end portion of the medical device 5400 described below with respect to FIGS. 16-20.

As shown, for example, in FIG. 9, medical device 5400 also includes one or more end effector actuation elements 5420 (also referred to herein as “actuation elements”) that couple the proximal mechanical structure 5700 to the wrist assembly 5500 and end effector 5460. The actuation elements 5420 can be, for example, a cable, a band, rod, or the like. The medical device 5400 is configured such that select movements of the actuation elements 5420 produce rotation of the wrist assembly 5500 (i.e., pitch rotation) about a first axis of rotation A1 (see FIG. 9) (which functions as a pitch axis; the term pitch is arbitrary), yaw rotation of the end effector 5460 about a second axis of rotation A2 (see FIG. 9) (which functions as the yaw axis; the term yaw is arbitrary), a cutting or gripping rotation of the tool members of the end effector 5460 about the second axis of rotation A2, or any combination of these movements. Changing the pitch or yaw of the instrument 5400 can be performed by manipulating the actuation elements 5420 in a similar manner as described, for example, in U.S. Pat. No. 8,821,480 B2 (filed Jul. 16, 2008), entitled “Four-Cable Wrist with Solid Surface Cable Channels,” which is incorporated herein by reference in its entirety. Thus, the specific movement of each of the drive elements to accomplish the desired motion is not described below.

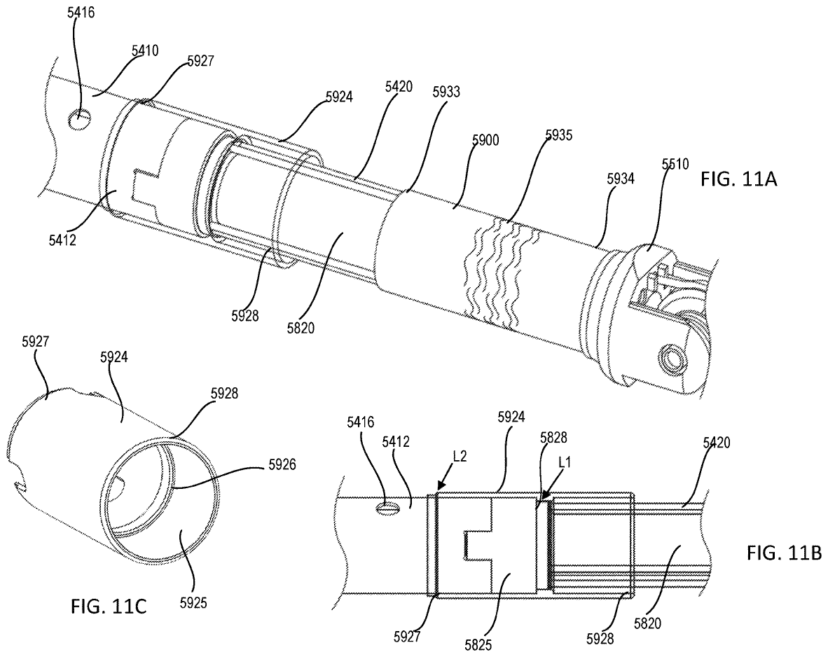

The inner shaft 5410 includes a proximal end portion 5411 that is coupled to the fluid routing structure 5919, and a distal end portion 5412 that is coupled to a beam 5810 of the distal force sensor unit 5800 (see, FIGS. 11A and 11B). The beam 5810 can include or have coupled thereto one or more strain sensors 5830 (see FIG. 13) to measure forces imparted on the surgical instrument in the x and y directions during a surgical procedure. The proximal end portion 5411 of the inner shaft 5410 is coupled to the proximal mechanical structure 5700 in a manner that allows translational movement of the inner shaft 5410 along a z-axis direction relative to the proximal mechanical structure 5700. The inner shaft 5410 also defines a lumen (not shown) and/or multiple passageways through which the actuation elements 5420 and other components (e.g., electrical wires, ground wires, or the like) can be routed from the proximal mechanical structure 5700 to the wrist assembly 5500.

The wrist assembly 5500 includes a proximal first link 5510 and a distal second link 5610. The first link 5510 is coupled to the second link 5610 such that the second link 5610 can rotate relative to the first link 5510 about the first axis of rotation A₁ (which functions as the pitch axis, the term pitch is arbitrary). The proximal first link 5510 includes a proximal portion that is coupled to a distal end portion 5812 of the beam 5810. The distal second link 5610 is coupled to the end effector 5460 such that the end effector 5460 can rotate about the second axis of rotation A2 (see FIG. 9). In this embodiment, the end effector 5460 includes first tool member 5462 and a second tool member 5482 forming jaws for engaging, grasping and/or manipulating tissue during a surgical procedure. The end effector 5460 is operatively coupled to the proximal mechanical structure 5700 such that the tool members 5462 and 5482 rotate relative to inner shaft 5410 about the first axis of rotation A1. Although the end effector 5460 includes tool members 5462, 5482 that are jaws or grippers (which can be used as needle drivers, for example), in alternative embodiments, the end effector 5460 can include other types of tools such as a cutter, an energized tool member that is used for cauterization or electrosurgical procedures, etc.

During a medical procedure, the tools 5462, 5482 of the end effector 5460 can contact anatomical tissue, which may result in x, y, or z direction forces (see, e.g., x, y, and z axes directions shown in FIG. 9) being imparted on the tools 5462, 5482. The strain sensor(s) 5830 can measure strain in the beam 5810 during operation of the medical device 5400. The measured beam strain can be used to determine forces imparted on the tools 5462, 5482 in the x- and y-axis directions. These x- and y-axis forces are transverse (e.g., perpendicular) to the z-axis (which is parallel or collinear with a center axis of the beam).

The proximal mechanical structure 5700 includes a chassis that supports or contains components configured to actuate the actuation elements 5420, which causes one or more components of the surgical instrument to move, such as, for example, the wrist assembly 5500 or the tools 5462, 5482. The actuation elements 5420 extend from the proximal mechanical structure 5700 to the wrist assembly 5500 and drive pulleys 5467, 5487 of the tool members 5462, 5482 of the end effector 5460 (see FIG. 9). As shown in FIG. 8, the proximal mechanical structure 5700 also includes an instrument support structure that includes a base 5770. In other embodiments, various support structures optionally may be used, such as a chassis, a frame, a bed, a unitized surrounding outer body of the proximal mechanical structure, and the like.

The outer shaft 5910 can be any suitable elongated shaft that can be disposed over the inner shaft 5410 and includes a proximal end portion 5911 that is coupled to the fluid routing structure 5919 and a distal end portion 5912. The outer shaft 5910 defines a lumen between the proximal end portion 5911 and the distal end portion 5912. The inner shaft 5410 extends within the lumen of the outer shaft 5910 and can move relative to the outer shaft 5910. For example, the inner shaft 5410 can translate longitudinally in a direction parallel to a center axis of the inner shaft 5410.

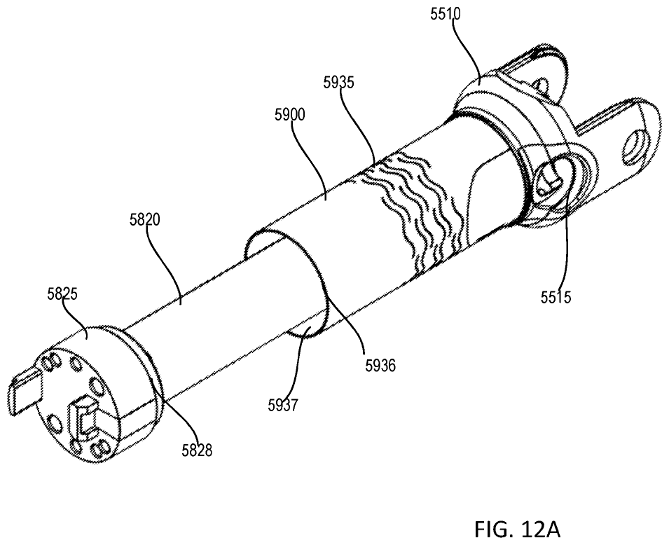

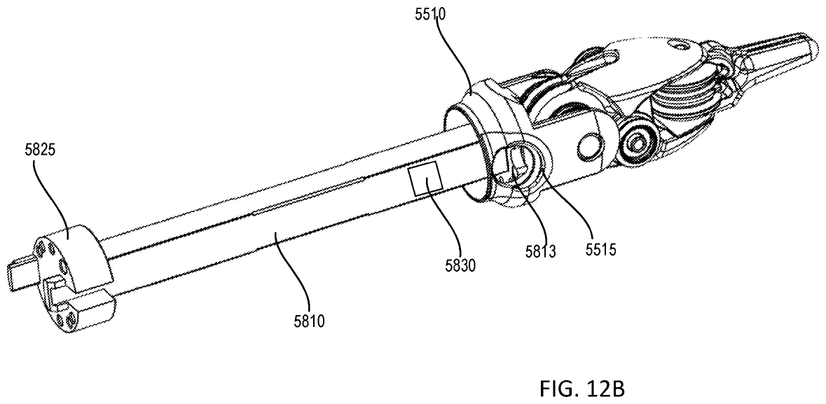

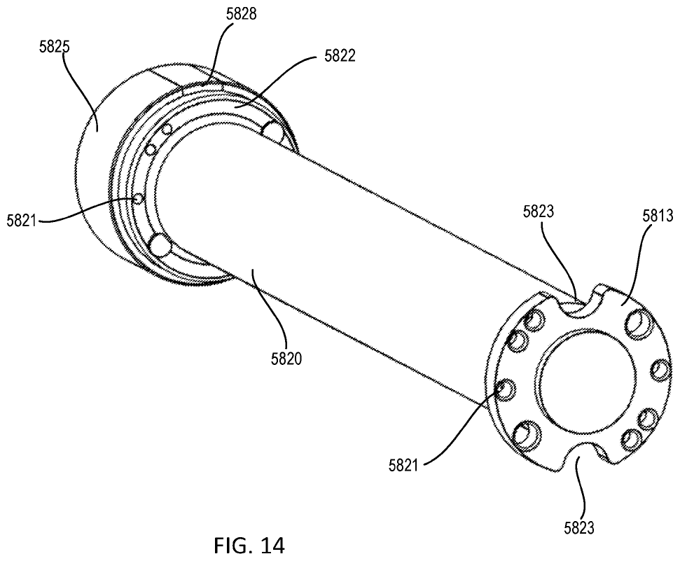

As shown, for example in FIGS. 12A, 12B, and 13, an overmold component 5820 is disposed on the beam 5810 to protect the beam 5810 and strain sensor 5830 from damage and exposure to bodily fluids and material. The beam 5810 also includes an anchor 5825 at a proximal end of the beam 5810 to which the overmold 5820 is coupled. FIG. 12B shows the beam 5810 without the overmold 5820 for illustration purposes. The anchor 5825 is coupled to the distal end portion 5412 of the inner shaft 5410. The anchor 5825 includes openings 5821 (see, e.g., FIG. 14) through which actuation elements 5420 and other wires can be routed and a shoulder 5828 that is coupled to the distal bushing 5924 described below. The distal end portion 5812 of the beam 5810 includes a connector 5813 that includes openings 5821 for routing the actuation elements 5420 and other wires. The connector 5813 also defines cutouts 5823. The connector 5813 is used to couple the beam 5810 to the shroud 5900 and to the first link 5510, as described in more detail below. The overmold 5820 also provides a seal 5822 for the actuation elements 5420 extending through the openings 5821 to prevent fluids and other material from passing through the openings 5821 proximally.

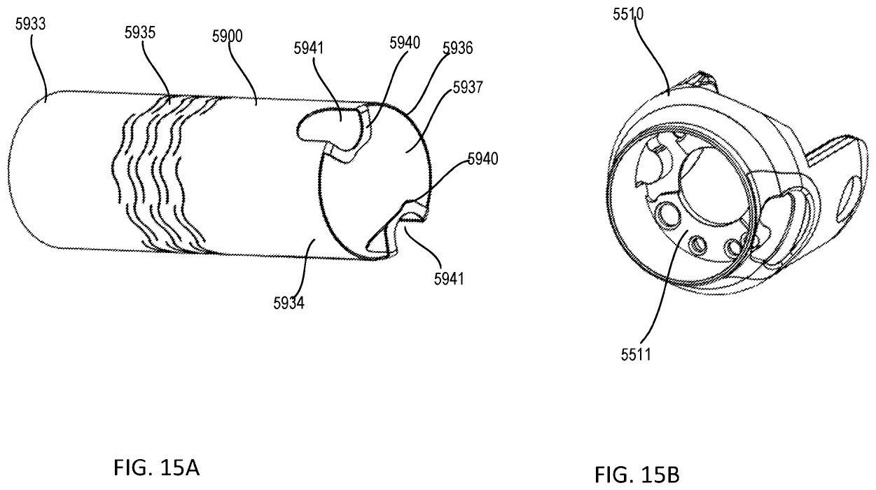

The shroud 5900 includes a distal end portion 5934 coupled to the first link 5510 and to the beam 5810, and a proximal end portion 5933 that extends proximally over the beam 5810 (and overmold 5820). As shown in FIGS. 13, 15A, and 15B, the shroud 5900 includes a pair of tabs 5940, and openings 5941. The tabs 5940 couple the shroud 5900 to the connector 5813 of the beam 5810 and to the first link 5510. More specifically, the tabs 5940 are captured or sandwiched between the first link 5510 and the connector 5813 such that the tabs 5940 abut a distal cable routing structure 5511 within the first link 5510 (see FIG. 15B) and a portion of the connector 5813, securing the shroud 5900 to the distal end portion 5812 of the beam 5810 and to the first link 5510. This eliminates the need for additional attachment mechanisms such as, for example, welding for securing the shroud 5900.

As best shown in FIG. 15A, the shroud 5900 is tubular and defines an interior volume 5937. Shroud 5900 also optionally defines multiple slits 5935 through a wall 5936 of the shroud 5900. As described above, while medical device 5400 is illustrated as including slits 5935 in the various views of FIGS. 8-20, the slits 5935 are merely optional design features and are not required to be included. The shroud 5900 may or may not define or include the multiple slits 5935 in any of the embodiments as described herein. The slits 5935 have a wavy or curved shape, and have a width sized to prevent sutures catching in the slits 5935. This can be particularly advantageous in applications where the end effector is a needle driver for use in suturing during various procedures. With such a configuration of the slits 5935, even during deformation of the shroud, the shroud 5900 can avoid any undesirable pinching or catching of the suture. The slits 5935 provide enhanced flexibility and bendability of the shroud 5900. In some embodiments, the slits 5935 have a width of less than about 0.10 mm. The shroud 5900 is positioned to cover and protect the strain sensor 5830 on the beam 5810, the actuation elements 5420, wires, etc. that may be located at the distal end portion of the medical device 5400. The shroud 5900 can be formed with a super elastic, shape-memory material, such as, for example, Nitinol alloy. Thus, the shroud 5900 can be bent or deformed and revert back to a biased linear configuration as shown, for example, in FIGS. 9, 10, 11A, 12A, and 15A. For example, the shroud 5900 is resiliently deformable radially inward and resiliently bendable radially inward during use of the medical device 5400, as described in more detail below. The super elastic material of the shroud 5900 provides more tolerance for misalignment between the shroud 5900 and the inner shaft 5410 due to its flexibility, which also provides more sensing range for the force sensor unit 5800. The shroud 5900 can also be formed with a relatively thin wall thickness to enhance the sensing range and to provide more clearance to other components for cleaning the medical device 5400 as described in more detail below. For example, in some embodiments, the wall thickness of the shroud 5900 can be about 0.076 mm (0.003 inches). The super elastic material, the thin wall thickness and the slits provide more flexibility to the shroud 5900 allowing the shroud 5900 to also deflect or deform during cleaning for better flow of cleaning fluid within the medical device 5400.

Referring to FIGS. 10, 11A-11C, the outer shaft 5910 extends distally over and surrounds the distal end portion 5412 of the inner shaft 5410, a portion of the beam 5810 (and overmold 5820), and a portion of the shroud 5900. As shown, for example, in FIG. 10, a distal end portion 5912 of the outer shaft 5910 is positioned at a contact region 5930 between the distal end portion 5912 of the outer shaft 3910 and the shroud 5900 and the contact region 5930 is associated with the location of the multiple slits 5935 (e.g., the contact region is at a same or generally same location as the slits). The outer shaft 5910 extending partially over the slits 5935 also minimizes exposure of the slits 5935 to fluids and other bodily materials during use. As described above, the inner shaft 5410, which is coupled to the anchor 5825 at the proximal end portion 5811 of the beam 5810, can translate within the outer shaft 5910 within a range of motion defined between a proximal range of motion limit and a distal range of motion limit, and within this range of motion of the inner shaft 5410, the proximal end portion 5933 of the shroud 5900 remains within or surrounded by the outer shaft 5910. In other words, as the inner shaft 5410, beam 5810, shroud 5900, and first link 5510 translate proximally and distally relative to the outer shaft 5910, the proximal end portion 5933 of the shroud 5900 remains surrounded by the outer shaft 5910.

As shown in FIGS. 9, 10, and 11A-11C, a proximal end portion 5927 of the distal bushing 5924 is coupled to the distal end portion 5412 of the inner shaft 5410 and a distal end portion 5928 extends distally over the proximal end portion 5811 of the beam 5810. The distal bushing 5924 includes an interior volume 5925 and a circumferential interior protrusion 5926 (see FIG. 11C). The interior protrusion 5926 is captured by the shoulder 5828 (see FIG. 14) of the anchor 5825 at location L1 in FIG. 11B, which is coupled to the distal end portion 5412 of the inner shaft 5410. The proximal end portion 5927 of the distal bushing 5924 is coupled to the distal end portion 5412 of the inner shaft 5410 with, for example, a weld at location L2 in FIG. 11B. The distal bushing 5924 provides support to the distal end portion 5912 of the outer shaft 5910, provides a low friction bearing surface for relative motion between the inner shaft 5410 and the outer shaft 5910, creates an insufflation barrier to prevent air exiting the surgical space, and is used during cleaning of the medical device as described below.

During operation of the medical device, the beam 5810 can bend or deflect due to outside forces imparted on the distal end portion of the medical device 5400 (e.g., on the wrist assembly 5500 or end effector 5460). For example, the beam 5810 can bend radially outward. Because the shroud 5900 is coupled to the beam 5800, as the beam 5800 bends due to these outside forces, the shroud 5900 will move with the beam 5810 until it contacts the distal end portion 5912 of the outer shaft 5910 at for example the contact region 5930. The material, thin wall thickness and/or slits of the shroud 5900 allow the shroud 5900 to resiliently deform radially inward and/or resiliently bend along the longitudinal axis of the shroud 5900 as it contacts the outer shaft 5910. In doing so, the lateral deflection of the beam 5810 is limited by the contact between the shroud 5900 and the outer shaft 5910. As stated above, the shroud 5900 can then revert to its biased linear shape when the shroud 5900 is no longer in contact with the outer shaft 5910.

The components of the medical device 5400 also provide access to the distal end portion of the medical device 5400 for cleaning purposes. As shown, for example, in FIGS. 9, 10, 12A, 12B and 13, the proximal first link 5510 includes a fluid port 5515 through which fluid can be introduced into the distal end portion of the medical device 5400 to clean interior components of the medical device 5400. The fluid port 5515 is fluid communication with the openings 5941 of the shroud 5900 and the cutouts 5823 of the connector 5813 of the beam 5810. Thus, as shown in FIG. 10 by the arrows FF, when fluid is introduced through the fluid port 5515, the fluid flows proximally through the openings 5941 and cutouts 5823, into the interior volume of the shroud 5900, where the fluid is directed by the shroud 5900 into the interior volume 5925 of the distal bushing 5924. The fluid is prevented from flowing past the anchor 5825 and distal end of the distal bushing 5924 and is routed back distally to an exit location at the distal end of the medical device 5400 as shown by arrows BF. For example, most of the fluid will flow back distally in the gap between an outer surface of the shroud 5900 and the inner surface 5917 of the outer shaft 5910, and some fluid may flow back into the interior volume of the shroud 5900 and distally to the distal end of the medical device 5400.

Thus, the fluid can flow within the medical device 5400 along the overmold 5820 of the beam 5810 and along the actuation elements 5420. The outer shaft 5910 is positioned to help contain the fluid within the medical device 5400 as the fluid flows between the proximal end portion 5933 of the shroud 5900 and the distal end 5928 of the distal bushing 5924. The fluid port 5515 is positioned on the first link 5510 in a location where it can be easily accessed and connected to a fluid source. In some embodiments, the fluid port 5515 can be configured to be coupled to a luer connector of a fluid source. In some embodiments, an adapter can be used to couple a fluid source to the fluid port 5515.

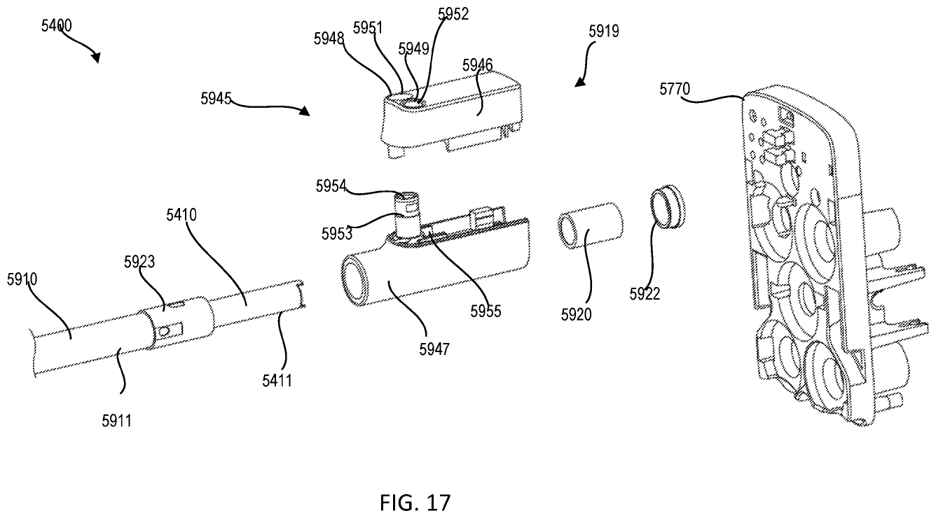

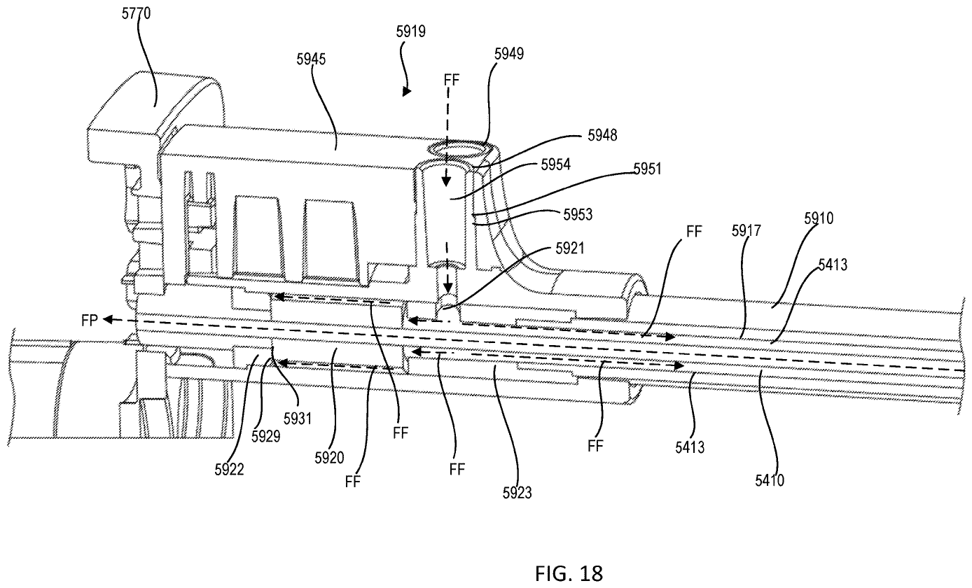

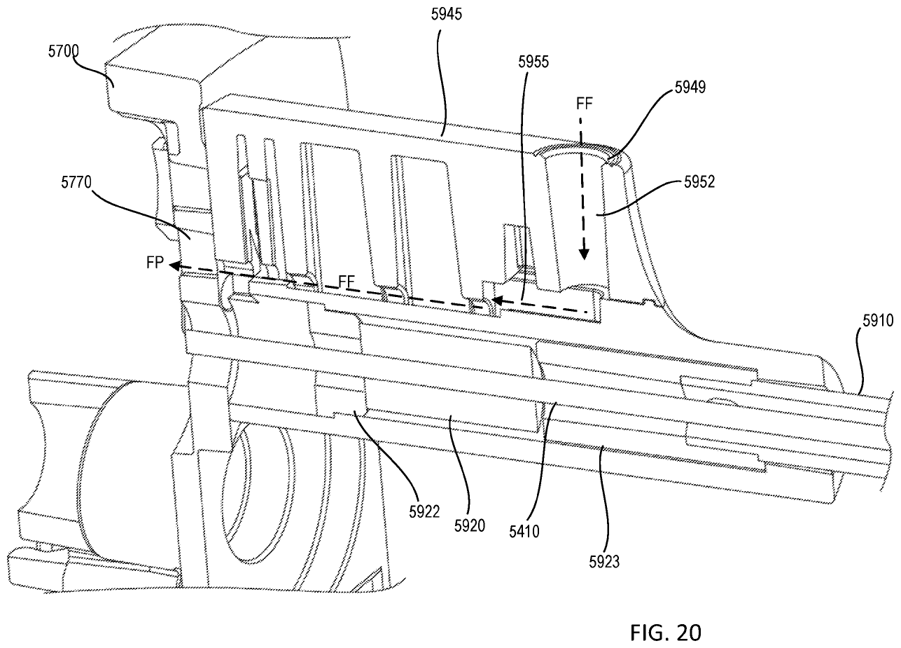

FIGS. 16-20 illustrate the fluid routing structure 5919 and components at a proximal end portion of the medical device 5400 to provide access for cleaning the interior components at the proximal end portion of the medical device 5400. As shown for example, in the exploded view of FIG. 17, the fluid routing structure 5919 includes a flush port structure 5945, a flow restriction tube 5920, and a flow restriction tube stop 5922. The fluid routing structure 5919 also includes a coupler 5923 (see FIGS. 18-20) within an interior of the fluid routing structure 5919. The flush port structure 5945 includes a first component 5946 coupled to a second component 5947. The first component 5946 includes a first flush port 5948, a second flush port 5949, a passageway 5351 in fluid communication with the first flush port 5948 and first flush channel 5952. The second component 5947 includes an extension tube 5953 that is received within the passageway 5951 (see FIG. 18) and that defines a second flush channel 5954. The second component 5947 also defines an interior region 5955. When the first component 5946 is coupled to the second component 5947, the first flush port 5948 is in fluid communication with the second flush passageway 5954 (as shown in FIG. 18), and the second flush port 5949 and first flush channel 5952 is in fluid communication with the interior region 5955 (as shown in FIG. 20).

As shown in FIGS. 18-20, the inner shaft 5410 extends within at least a portion of the fluid routing structure 5919 and the flow restriction tube 5920 and the flow restriction tube stop 5922 each surround a portion of the inner shaft 5410 within the flow restriction structure 5919. The flow restriction tube stop 5922 is fixedly positioned proximally of the flow restriction tube 5920. The flow restriction tube 5920 can translate proximally and distally relative to the inner shaft 5410 as described above for medical device 4400. When the flow restriction tube 5920 translates proximally, the flow restriction tube stop 5922 limits the translation of the flow restriction tube 5920 in the proximal direction. In other words, the flow restriction tube 5920 can move from a first position in which a proximal end portion 5931 of the flow restriction tube 5920 is spaced apart from a distal end 5929 of the flow restriction tube stop 5922 (not shown in FIG. 18020) (see, e.g., flow restriction tube 4920 in FIG. 7A) and a second position in which the proximal end 5931 of the flow restriction tube 5920 contacts the distal end 5929 of the flow restriction tube stop 5922, as shown in FIGS. 18-20.

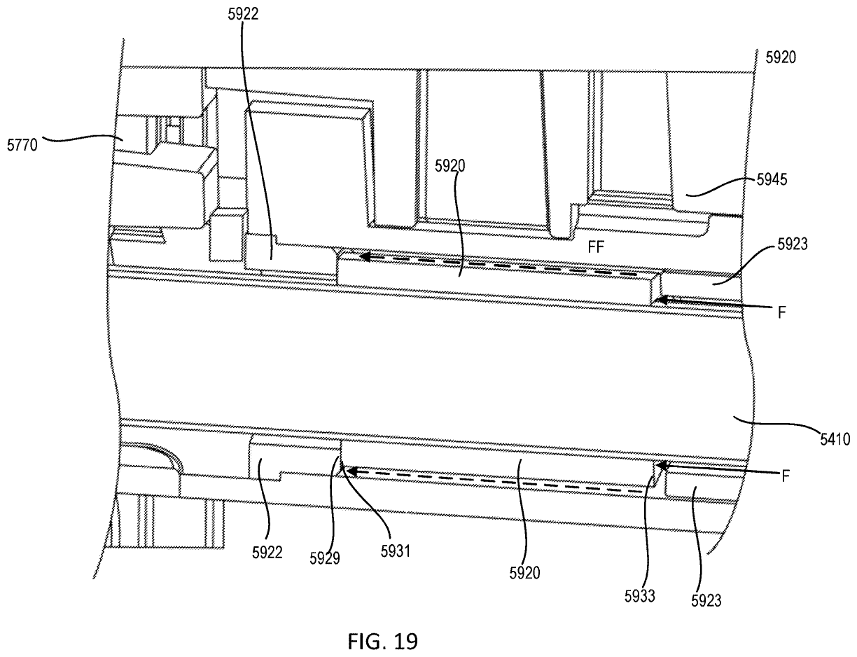

The fluid routing structure 5919 allows for the introduction of fluid into the medical device 5400 to clean interior components of the medical device 5400. Fluid can be introduced through the first fluid port 5948, the second fluid port 5949 or both. When a fluid is introduced through the first fluid port 5948, the fluid will flow through the first flush channel 5954, through the opening 5921 of the coupler 5923 and can flow both proximally and distally as shown by arrows FF in FIGS. 18 and 19. The fluid flowing distally can flow in a gap between an outer surface 5413 of the inner shaft 5410 and an inner surface 5917 of the outer shaft 5910. The fluid can flow within the gap along the outer surface 5413 of the inner shaft 5410 (as shown by arrow FD in FIG. 10) to a distal end portion of the medical device 5400 where the fluid can exit the medical device 5400. For example, at the distal end portion 5412 of the shaft 5410, the fluid can exit or enter through one or more return openings 5416 (see, e.g., opening 5416 shown in FIGS. 10, 11A and 11B) and flow in a reverse or return direction (proximally) within an inner volume of the shaft 5410 (as shown by arrow FP in FIG. 10 and FIG. 18), thus cleaning the interior of the shaft 5410. The fluid flowing in the proximal direction can impart a fluid pressure force F (see FIG. 19) on a distal end 5933 of the flow restriction tube 5920 and cause the flow restriction tube 5920 to translate proximally until the proximal end 5931 of the flow restriction tube 5920 contacts the distal end 5929 of the flow restriction tube stop 5922. The fluid can flow around an outer surface of the flow restriction tube 5920 but is prevented from traveling beyond the flow restriction tube stop 5922 in a proximal direction. As shown in FIG. 20, when a fluid is introduced through the second fluid port 5949, the fluid flows through the first flush channel 5952, into the interior region 5955, and proximally into the proximal mechanical structure 5700 (e.g., to clean additional portions of the medical device 5400).

While various embodiments have been described above, it should be understood that the various embodiments have been presented by way of example only and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a telesurgical system that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the components of a surgical instrument as described herein can be constructed from any material, such as medical grade stainless steel, nickel alloys, titanium alloys or the like. Further, any of the links, tool members, beams, shafts, connectors, cables, or other components described herein can be constructed from multiple pieces that are later joined together. For example, in some embodiments, a link can be constructed by joining together separately constructed components. In other embodiments however, any of the links, tool members, beams, shafts, connectors, cables, or components described herein can be monolithically constructed.

Although the instruments are generally shown as having an axis of rotation of the tool members (e.g., axis A2) that is normal to an axis of rotation of the wrist member (e.g., axis A1), in other embodiments any of the instruments described herein can include a tool member axis of rotation that is offset from the axis of rotation of the wrist assembly by any suitable angle. Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device comprising:
a shaft comprising a distal end portion;
a beam comprising a proximal end portion and a distal end portion, the proximal end portion of the beam being coupled to the distal end portion of the shaft;
a body coupled to the distal end portion of the beam, the body comprising a fluid port; and
a shroud comprising a proximal end portion, a distal end portion, and an inner wall between the proximal end portion and the distal end portion of the shroud;
wherein the distal end portion of the shroud is coupled to the body;
wherein the proximal end portion of the shroud is located between the distal end portion of the shaft and the body; and
wherein the inner wall of the shroud defines an interior volume in fluid communication with the fluid port such that fluid introduced into the fluid port flows through the interior volume of the shroud and is directed proximally toward the distal end portion of the shaft.

2. The medical device of claim 1, wherein:
the medical device includes an end effector actuator element;
the end effector actuator element extends through the distal end portion of the shaft and exits the distal end portion of the shaft at an exit location; and
fluid introduced into the fluid port flows through the interior volume of the shroud, is directed proximally along the end effector actuator element, and is directed against the exit location.

3. The medical device of claim 1, wherein:
the medical device further comprises a bushing;
the bushing comprises a proximal end portion and a distal end portion;
the proximal end portion of the bushing is coupled to the distal end portion of the shaft; and
the distal end portion of the bushing is located between the distal end portion of the shaft and the proximal end portion of the shroud.

4. The medical device of claim 3, wherein:
the bushing defines an interior volume between the distal end portion of the shaft and the distal end portion of the bushing; and
fluid introduced into the fluid port flows through the interior volume of the shroud and proximally into the interior volume of the bushing.

5. The medical device of claim 3, wherein:
the medical device includes an end effector actuator element;
the bushing defines an interior volume between the distal end portion of the shaft and the distal end portion of the bushing;
the end effector actuator element extends through the distal end portion of the shaft, exits the distal end portion of the shaft at an exit location, and extends through the interior volume of the bushing; and
fluid introduced into the fluid port flows through the interior volume of the shroud, is directed proximally along the end effector actuator element, is directed into the interior volume of the bushing, and is directed against the exit location.

6. The medical device of claim 1, wherein:
the shaft is an inner shaft;
the medical device further comprises an outer shaft surrounding at least a portion of the inner shaft;
the outer shaft comprises a distal end;
the shroud comprises a plurality of slits positioned at a contact region between the shroud and the distal end of the outer shaft; and
contact between the shroud and the distal end of the outer shaft limits lateral deflection of the distal end portion of the beam.

7. The medical device of claim 6, wherein:
a longitudinal axis of the shroud is defined between the proximal end portion and the distal end portion of the shroud; and
at the contact region, the shroud is resiliently deformable radially inward and is resiliently bendable along the longitudinal axis of the shroud.

8. The medical device of claim 6, wherein:
each slit of the plurality of slits is shaped, sized, or shaped and sized to restrict capture of a surgical suture.

9. The medical device of claim 1, wherein:
the shaft is an inner shaft;
the medical device further comprises an outer shaft surrounding at least a portion of the inner shaft and surrounding the proximal end portion of the shroud;
the inner shaft translates within the outer shaft within a range of motion defined between a proximal range of motion limit and a distal range of motion limit; and
the proximal end portion of the shroud remains within the outer shaft within the range of motion of the inner shaft.

10. A medical device comprising:
a fluid routing structure;
a shaft extending within at least a portion of the fluid routing structure;
a flow restriction tube surrounding the shaft within the fluid routing structure; and
a flow restriction tube stop positioned proximally of the flow restriction tube;
wherein pressure from a fluid introduced against the flow restriction tube causes the flow restriction tube to translate proximally with reference to the shaft until the flow restriction tube contacts the flow restriction tube stop; and
wherein contact between the flow restriction tube and the flow restriction tube stop restricts the fluid from traveling proximally past the flow restriction tube stop.

11. The medical device of claim 10, wherein:
the shaft comprises an exterior surface;
the fluid routing structure comprises a flush port structure;
a flush port is defined in the flush port structure; and
the fluid is introduced through the flush port, is thereafter routed proximally along the exterior surface of the shaft, and is thereafter introduced against the flow restriction tube.

12. The medical device of claim 10, wherein:
the flow restriction tube is positioned to translate along a length of the shaft within the fluid routing structure.

13. The medical device of claim 11, wherein:
the shaft is an inner shaft;
the medical device further comprises an outer shaft and a coupler;
the outer shaft comprises a proximal end portion coupled to the coupler;
the coupler is coupled to the fluid routing structure; and
the coupler comprises a port in fluid communication with the flush port of the flush port structure.

14. The medical device of claim 13, wherein:
the outer shaft comprises an interior surface; and
the fluid is introduced through the flush port and is thereafter routed through the port of the coupler and distally between the exterior surface of the shaft and the interior surface of the outer shaft.

15. The medical device of claim 11, wherein:
the flush port is a first flush port;
the medical device further comprises a proximal mechanical structure coupled to the fluid routing structure;
the flush port structure comprises a second flush port; and
a fluid introduced into the second flush port is directed proximally to a location within the proximal mechanical structure.

16. The medical device of claim 13, wherein:
the flow restriction tube comprises a proximal end and a distal end, and a longitudinal axis of the flow restriction tube is defined between the proximal and distal ends of the flow restriction tube;
the coupler comprises a proximal end; and
the flow restriction tube is movable along the longitudinal axis of the flow restriction tube between the proximal end of the coupler and the flow restriction tube stop.

17. A medical device comprising:
a shaft comprising a distal end portion;
a beam comprising a proximal end portion and a distal end portion, the proximal end portion of the beam being coupled to the distal end portion of the shaft;
a body coupled to the distal end portion of the beam;
a shroud comprising a proximal end portion, a distal end portion, and an inner wall between the proximal end portion and the distal end portion of the shroud; and
a bushing comprising a proximal end portion and a distal end portion, the proximal end portion of the bushing is coupled to the distal end portion of the shaft and
the distal end portion of the bushing is located between the distal end portion of the shaft and the proximal end portion of the shroud.
wherein the distal end portion of the shroud is coupled to the body;
wherein the proximal end portion of the shroud is located between the distal end portion of the shaft and the body; and
wherein the inner wall of the shroud defines an interior volume configured to receive a fluid that flows through the interior volume of the shroud and is directed proximally toward the distal end portion of the shaft.

18. The medical device of claim 17, wherein:
the bushing defines an interior volume between the distal end portion of the shaft and the distal end portion of the bushing;
the body comprises a fluid port; and
fluid introduced into the fluid port flows through the interior volume of the shroud and proximally into the interior volume of the bushing.

19. The medical device of claim 17, wherein:
the medical device includes an end effector actuator element;
the bushing defines an interior volume between the distal end portion of the shaft and the distal end portion of the bushing;
the end effector actuator element extends through the distal end portion of the shaft, exits the distal end portion of the shaft at an exit location, and extends through the interior volume of the bushing; and
fluid introduced into the interior volume of the shroud, is directed proximally along the end effector actuator element, is directed into the interior volume of the bushing, and is directed against the exit location.

20-27. (canceled)
